(19) United States
(12) Patent Application Publication
(10) Pub. No.: US 2022/0356134 A1
(43) Pub. Date: Nov. 10, 2022

(54) PROCESS FOR PREPARING ALKYLENE GLYCOL FROM A CARBOHYDRATE SOURCE COMPRISING HEMICELLULOSE, CELLULOSE AND LIGNIN

(71) Applicant: Avantium Knowledge Centre B.V., Amsterdam (NL)

(72) Inventors: Benjamin McKay, Amsterdam (NL); Gerardus Johannes Maria Gruter, Amsterdam (NL); Martijn Kersbulck, Amsterdam (NL)

(73) Assignee: Avantium Knowledge Centre B.V., Amsterdam (NL)

(21) Appl. No.: 17/624,416

(22) PCT Filed: Jul. 13, 2020
(86) PCT No.: PCT/EP2020/069747

§ 371 Date: Jan. 3, 2022

(30) Foreign Application Priority Data
Jul. 31, 2019 (EP) 19189459.1

(51) Int. Cl. C07C 29/132; C07C 29/60

(52) U.S. Cl. CPC C07C 29/132; C07C 29/60

(57) ABSTRACT

A process for preparing alkylene glycol from particulate matter comprising hemicellulose, cellulose and lignin, which process comprises the steps of subjecting a reactor comprising such particulate matter to a two-stage hydrolysis in the presence of hydrochloric acid to hydrolase the hemicellulose and cellulose in the particulate matter to saccharides, followed by subjecting the obtained hydrolysates to a catalytic conversion with hydrogen and in the presence of a catalyst system to a product comprising one or more alkylene glycols.

The invention relates to a process for preparing alkylene glycol (e.g. ethylene glycol and/or propylene glycol) from a carbohydrate source comprising hemicellulose, cellulose and lignin. The process comprising preparing a mixture of (dissolved) pentoses and/or hexoses and its oligomers by a 2-stage acid hydrolysis of matter comprising hemicellulose, cellulose and lignin, and a further stage which converts such pentose and/or hexose saccharides into alkylene glycols by catalytic conversion with hydrogen.

### BACKGROUND OF THE INVENTION

Alkylene glycols such as ethylene glycol and propylene glycol are valuable products or intermediates in chemical industry, as such compounds are used in various chemical processes. Traditionally, alkylene glycols are produced from fossile sources. More recently, there is ongoing research to produce alkylene glycols from renewable sources.

In this connection, CN 102643165 describes a process for producing ethylene glycol and propylene glycol from soluble sugars or starch. This reference is silent as to the sources of soluble sugars or starch, and using soluble sugars as a source for producing chemicals may compete in an undesired fashion with the human food chain. Similarly, U.S. Pat. No. 7,960,594 discloses a process in which ethylene glycol is produced from cellulose. It states in the text that the cellulose may be obtained from forestry residues or other sources of cellulose from material unsuitable for human consumption, yet it does not provide any details as to how such cellulose can be obtained from such sources. In WO 2016/114661 it is stated that ethylene glycol may be obtained from a carbohydrate source, which carbohydrate source may be the hydrolysis product of lignocellulosic biomass, without giving any further particulars. Lignocellulosic biomass is generally seen as a material not suitable for human consumption. Examples of such lignocellulosic material include wood, straw, nutshells, corn stover and bagasse.

In order to be able to produce alkylene glycols from renewable sources which do not compete with the human food chain or are not used as such in the human food chain or compete with the human food chain it is desired that there is a process for producing alkylene glycols from lignocellulosic biomass.

Several processes have been studied in the past to obtain useful materials from lignocellulosic material. An example of such is the Bergius-Rheinau process. In the Bergius-Rheinau process solid lignocellulosic material, such as wood, is treated with a concentrated hydrochloric acid composition. Such hydrochloric acid treatment may result in (partial) hydrolysis of the cellulose and hemicellulose and thus give a hydrolysate and a residue that consists for a large part of lignin. From the hydrolysate of cellulose and hemicellulose saccharides (typically mono- and oligosaccharides) may be obtained, which saccharides can be used in further conversion processed to make e.g. ethanol, ethyleneglycol and other (base) chemicals. This Bergius-Rheinau process has been described by F. Bergius, Current Science , Vol. 5, No. 12 (June 1937), pp. 632-637 and the hydrolysis step is in essence a one-stage hydrolysis using hydrochloric acid of 40%, which hydrolyses both hemicellulose and cellulose. A hydrolysate obtained with such process contains both saccharides originating from hemicellulose (e.g. xylose, arabinose, mannose, glucose and their oligomers) and cellulose (mainly glucose and its oligomers).

The hydrolysis of the hemicellulose and cellulose may also be effected in two-stages: a first stage hydrolyzing mainly hemicellulose and a second stage hydrolyzing mainly cellulose. The advantage of such is that the resulting saccharide fractions can be obtained separately, which provides more options for adding value to the resulting hydrolysates. An example of such a two-stage hydrolysis of lignocellulosic biomass is described in U.S. Pat. No. 294,577, which is a Bergius-Rheinau type process modified by the patentee (Riehm). The process disclosed therein uses a hydrochloric acid solution of 34-37% for hydrolyzing the hemicellulose fraction of the lignocellulosic biomass (e.g. pinewood sawdust) first (named prehydrolysis) followed by a hydrolysis of the cellulose fraction of the remaining material using a hydrochloric acid solution of 40-42% (named main hydrolysis). GB827921 discloses a process for producing sugars from cellulosic materials containing cellulose, lignin and hemicellulose, by contacting such cellulosic material with concentrated hydrochloric acid, and obtaining the hydrolysate of hydrolysed hemicellulose and optionally hydrolysed cellulose. The hydrolysates are reported to be suitable for use as animal feed or fermentation material.

A more recent example of such two-stage hydrolysis is disclosed in WO2016/082816. In the process in this reference in a vertical reactor filled with vegetable biomass particle, hydrochloric acid of 35-37% is fed from below into the reactor to effect hydrolysis of the hemicellulose, followed by feeding at the bottom of the reactor a 40-42% hydrochloric acid solution (to effect hydrolysis of cellulose) which displaces the 35-37% hydrochloric acid solution. It is stated that the flowspeed of the hydrochloric acid should be such that displacement of the lower concentrated acid by the higher concentrated acid would lead to minimal mixing of both acid fractions, without giving any further indication as to how this needs to be effected.

Hence, there is a need for a process for preparing alkylene glycol (e.g. ethylene glycol and/or propylene glycol) from a carbohydrate source comprising hemicellulose, cellulose and lignin (i.e. lignocellullosic matter). In this, it is desired that it is reasonably well possible to obtain such alkylene glycols from hydrolysis of the hemicellulose fraction (as such may give a mixture rich in ethylene glycol and propylene glycol) and/or the cellulose fraction (as such may give a mixture rich in ethylene glycol). Preferably, such process should be easy to control, be robust and not overly complex, time efficient, and yields (amount of hemicellulose and cellulose that can be converted into saccharides and its oligomers and obtained) should preferably high. The process should preferably also allow for different particulate lignocellulosic biomass sources, with different compositions.

### SUMMARY OF THE INVENTION

It has now been found that the above objective can be met, at least in part, by a process for preparing alkylene glycol from particulate matter comprising hemicellulose, cellulose and lignin, which process comprises the steps of subjecting a reactor comprising particulate matter comprising hemicellulose, cellulose and lignin and interstitial space to the following steps:
a. feeding to said reactor a first hydrochloric acid solution to hydrolyse at least part of the hemicellulose of said particulate matter by contacting the particulate matter with said first aqueous hydrochloric acid solution, which first aqueous hydrochloric acid solution has a hydrochloric acid concentration of between 30 wt. % and 42 wt. %, based on the weight amount of water and hydrochloric acid in such first aqueous hydrochloric acid solution, yielding a first remaining particulate matter and a first aqueous hydrolysate product solution;
b. feeding to said reactor a water-immiscible displacement fluid thereby displacing at least part of said first aqueous hydrolysate product solution from the interstitial space with said water-immiscible displacement fluid;
c. feeding to said reactor a second hydrochloric acid solution to hydrolyse at least part of the cellulose of the first remaining particulate matter by contacting the first remaining particulate matter with said second hydrochloric acid solution, which second hydrochloric acid solution has a hydrochloric concentration of between 40% and 51%, based on the weight amount of water and hydrochloric acid in the second hydrochloric acid solution whilst said second hydrochloric acid solution has a hydrochloric acid concentration which is the same or higher than the first hydrochloric acid solution added in step a., yielding a second remaining particulate solid material and a second aqueous hydrolysate product solution;
d. subjecting said first aqueous hydrolysate product and/or second aqueous hydrolysate product to a catalytic conversion with hydrogen and in the presence of a catalyst system to a product comprising one or more alkylene glycols,

wherein the catalytic conversion in step d. comprises a catalyst system comprising a tungsten compound, and at least one hydrogenolysis metal selected from the groups 8, 9 or 10 of the Periodic Table of the Elements.

The process of the present invention now allows alkylene glycols to be made from particulate matter comprising hemicellulose, cellulose and lignin (lignocellulosic biomass) using first a 2-stage hydrolysis process which has a step of using a displacement fluid in between which separates the hydrolysate from (mainly) hemicellulose from the hydrolysate of (mainly) cellulose, and thereafter the obtained hydrolysates (optionally after isolation and/or purification) may be used in a known process to prepare alkylene glycols out of the saccharides in the hydrolysates. In other words, it allows a process for the catalytic conversion of saccharides to do so on saccharides that can conveniently be obtained from lignocellulosic biomass which does not compete with the food chain for human consumption.

### DETAILED DESCRIPTION OF THE INVENTION

In the present invention, “particulate matter” herein in connection with the lignocellulosic biomass refers to material which is not liquid or gaseous but solid, and which is at the same time divided up in units such than when the reactor is filled with the particulate matter a bed is obtained which also contains interstitial space through which fluids can flow. For clarity, “particulate matter” herein covers fairly hard pieces such as woodchips and pieces of coconut shell but also fibrous material such as bagasse and particles made out if such.

“Interstitial space” herein means the voids in a reactor filled with particulate matter, or in other words the space inside the reactor but outside the particulate matter.

“Water-immiscible” herein means, in connection to the displacement fluid and displacement liquid, that such displacement fluid or displacement liquid has a solubility in water of less than 3 g displacement fluid (or displacement liquid) per litre of water, at 20° C. and atmospheric pressure. Preferably, such solubility is less than 2 g/L, even more preferably less than 1 g/L, under such conditions.

In the process of the present invention it may be preferred that there is an additional step after c. and before d. of separating the saccharides from the hydrolysate (isolating and optionally purifying the saccharides from the hydrolysate liquid). Suitable processes for obtaining a saccharide product from the pre-hydrolysate solution (i.e. the first hydrolysate product solution) and/or the main hydrolysate solution (i.e. the second hydrolysate solution) are described in for example WO2017/082723 and WO2016/099272. Preferably the pre-hydrolysate solution and/or the main hydrolysate solution is suitably first admixed with a carrier liquid, in which the saccharides are insoluble and that has a boiling point higher than that of water to obtain an aqueous admixture. Subsequently such aqueous admixture can be subjected to an evaporation step, to yield a vapor fraction comprising water and hydrochloric acid and a residue fraction comprising solid saccharides and the carrier liquid. The vapor fraction may advantageously be condensed, reconcentrated and recycled to the process to be used as a first or second hydrochloric acid solution. The residue fraction comprising solid saccharides and the carrier liquid can conveniently be recovered and passed to a separation vessel. Such a separation vessel can for example be a settling vessel or any other separator that is suitable to separate the saccharides from the carrier liquid. From the separation vessel a saccharide product can be obtained. In addition a stream of crude carrier liquid can be obtained that can be cleaned and recycled. Thus, preferably the process according to invention comprises one or more further steps wherein:

the first hydrolysate product solution and/or the second hydrolysate product solution is/are admixed with a carrier liquid, in which saccharides are insoluble and that has a boiling point higher than that of water to obtain an aqueous admixture;

the aqueous admixture is subjected to an evaporation step, to yield a vapor fraction comprising water and hydrochloric acid and a residue fraction comprising solid saccharides and the carrier liquid; and

the residue fraction comprising solid saccharides and the carrier liquid is passed to a separation vessel to obtain a saccharides product.

Steps a. to c. are preferably carried out in stationary flow-through bed, preferably multiple in series, in which the bed comprises the particulate matter. Contrary to this, step d. is preferably carried out in a reactor system comprising a continuously stirred tank reactor (CSTR). Hence, it may be preferred that there is an additional step of transferring the first and/or second aqueous hydrolysate product after step c. and prior to step d. to a continuously stirred tank reactor for effecting the catalytic conversion step d., as the reaction of d. can best be carried out in a reaction arrangement involving at least one CSTR, contrary to steps a.-c.

As to the process of step d., this is as such well known, e.g. from WO 2016/114661. In line with the process disclosed therein and for reasons set out in that reference, in the present invention the catalytic conversion in step d. comprises a catalyst system comprising a tungsten compound, and at least one hydrogenolysis metal selected from the groups 8, 9 or 10 of the Periodic Table of the Elements. Likewise, in the present invention it is preferred in the catalyst system the molar ratio of moles tungsten to moles hydrogenolysis metal is equal to or more than 1:1. As tungsten compound for the given reaction in such it is preferred that the tungsten compound has an oxidation state of at least 2+. More particularly, the tungsten compound herein is selected from the group consisting of: sodium tungstate (Na₂WO₄), tungstic acid (H₂WO₄), ammonium tungstate, ammonium metatungstate, ammonium paratungstate, tungstate compounds comprising at least one Group 1 or 2 element, metatungstate compounds comprising at least one Group 1 or 2 element, paratungstate compounds comprising at least one Group 1 or 2 element, tungsten oxide (WO₃), heteropoly compounds of tungsten, and combinations thereof.

It is preferred that hydrochloric acid (residues) in the first aqueous hydrolysate and/or second aqueous hydrolysate are removed from these hydrolysates prior to submitting them to step d. Hence, the first and/or second aqueous are preferably substantially free from hydrochloric acid prior to stubmitting them to step d. Additionally, the hydrolysates may contain part of the saccharides formed by the acid hydrolysis as oligomers, which are preferably hydrolysed to the corresponding monomer saccharides (i.e. pentoses and/or hexoses) prior to step d. Hence, such hydrolysation of oligomers to the corresponding monomers is preferably part of the process between steps c. and d. Next to this, an additional purification step of the first aqueous hydrolysate and/or second aqueous hydrolysate may be preferred. Also, part or all of the water from the first and/or second aqueous hydrolysate may be removed prior to step d.

As to the hydrogenolysis metal, also the same considerations apply as set out in WO 2016/114661. Hence, the hydrogenolysis metal is preferably from groups 8, 9 or 10 of the Periodic Table of the Elements is selected from the group consisting of Cu, Fe, Ni, Co, Pd, Pt, Ru, Rh, Ir, Os and combinations thereof. As to the physical state of such, it is preferred that the hydrogenolysis metal from the groups 8, 9 or 10 of the Periodic Table of the Elements is present in the form of a catalyst supported on a carrier. Preferred carriers in this connection are selected from the group supports, consisting of activated carbon, silica, alumina, silica-alumina, zirconia, titania, niobia, iron oxide, tin oxide, zinc oxide, silica-zirconia, zeolitic aluminosilicates, titanosilicates, magnesia, silicon carbide, clays and combinations thereof. A specifically preferred catalyst system comprises ruthenium on activated carbon.

In the present invention, it is preferred that the first hydrolysate obtained in step a. comprises pentoses and hexoses (i.e. C5- and C6-saccharides), which result from hydrolysis of hemicellulose. Likewise, it is preferred that the second aqueous hydrolysate product comprises hexoses (C6-saccharides) which result from cellulose hydrolysation.

Consequently, the resulting product preferably comprises ethylene glycol and/or propylene glycol. Hence, in the present invention it is preferred that the alkylene glycol is ethylene glycol and/or propylene glycol.

For the process according to the present invention, it is preferred that the first hydrochloric acid solution has a concentration of between 33 and 40 wt. %, based on the weight amount of water and hydrochloric acid in such first aqueous hydrochloric acid solution. More preferably such concentration is between 35 and 38 wt %, based on the weight amount of water and hydrochloric acid in such first aqueous hydrochloric acid solution. The concentration of the second hydrochloric acid solution used in the process according to the present invention is preferably between 40 and 46 wt. %, based on the weight amount of water and hydrochloric acid in such second aqueous hydrochloric acid solution, more preferably between 40 and 44 wt. %, based on the weight amount of water and hydrochloric acid in such second aqueous hydrochloric acid solution. However, the concentration of the second hydrochloric acid solution should be higher than that of the first. Hence, the lower range (e.g. 40-42%) of concentration given for the second hydrochloric acid can only be applied if the concentration of the first hydrochloric acid has a concentration of e.g. between 30 and 39 wt %, more likely 30-37 wt %. As already indicated by Bergius (publication under Background of the Invention), an advantage of hydrolysis using strong hydrochloric acid is that it can be carried out at ambient temperature and pressure. Hence, in the present invention it is preferred that the first hydrochloric acid and second hydrochloric acid added in steps a. and c. to the reactor are at a temperature of between 1 and 40° C., preferably between 5 and 30° C., and that the pressure in the reactors during steps a-c is about 0.1 MPa (atmospheric pressure).

During step (a) hemicellulose is being hydrolyzed and the resulting saccharides (typically a mixture of mono-, di-, and oligosaccharides) become dissolved in the first aqueous hydrochloric acid solution. Therefore, in addition to the water and the hydrochloric acid, the first aqueous hydrochloric acid solution may or may not contain other compounds such as for example dissolved saccharides. Similarly, during step (c) cellulose is being hydrolyzed and the resulting saccharides (typically a mixture of mono-, di-, and oligosaccharides) become dissolved in the second aqueous hydrochloric acid solution. Therefore, in addition to the water and the hydrochloric acid, the second aqueous hydrochloric acid solution may or may not contain other compounds such as for example dissolved saccharides. The process of subsequent steps a-c (and optionally an additional step with displacement fluid after c. and before d.) may be carried out in one or more reactors. Preferably, the process is carried out in at least two reactors in series wherein the reactors are at different stages in the process sequence of a-c. Also, multiple reactors may be used for step a and also for step c (and if desired also for step b, although such is less logical).

As mentioned herein before, a process has been developed as set out in WO2019149833, wherein the pre-hydrolysis (of mainly hemicellulose) and main hydrolysis (of mainly cellulose, using hydrochloric acid of greater concentration than for the pre-hydrolysis) are separated by using a displacement fluid. In the process of said reference, all three liquids (hydrochloric acid for pre-hydrolysis, displacement fluid, and hydrochloric acid for main-hydrolysis) flow through a reactor one after the other, which reactor contains lignocellulosic (biomass) particles. As stated under Summary of the invention, following step b. the water-immiscible displacement fluid displaces at least part of the first aqueous hydrolysate product solution obtained by step a. from the interstitial space with said water-immiscible displacement fluid. Similarly, the feeding to the reactor of said second hydrochloric acid solution in step c. may displace (and this is preferred) at least part of the water-immiscible displacement fluid from step b., thereby effecting removal of at least part of said water-immiscible displacement fluid from the interstitial space. Likewise, there may be and this is preferred) an additional step wherein after step c. and prior to step d. of feeding to said reactor a water-immiscible displacement fluid thereby displacing at least part of said second aqueous hydrolysate product solution from the interstitial space with said water-immiscible displacement fluid. The water-immiscible displacement fluid used for such additional step with displacement fluid (after c and before d.) may use a different water-immiscible displacement fluid or the same as was used for step b. It is preferred that these are the same. Additionally, it can be convenient to re-use the water-immiscible displacement fluid. In such a case, water-immiscible displacement fluid can be retrieved after step (c) and recycled to step (b). The water-immiscible displacement fluid retrieved after step (c) can optionally be purified and/or can optionally be stored in a displacement fluid storage vessel before being recycled to step (b). If there is an additional step (after c. and before d.) in which water-immiscible displacement fluid displaces at least part of the second aqueous hydrolysate product solution the same applies: it may rely on recycled displacement fluid.

As to the displacement fluid, it is preferred that it is a water-immiscible liquid (water-immiscibility as defined above). More preferably, the displacement fluid in the present process is a water-immiscible displacement liquid having a boiling temperature at 0.1 MPa of equal to or more than 50° C., more preferably equal to or more than 80° C. and even more preferably equal to or more than 100° C. Preferably, the water-immiscible displacement fluid has a melting temperature at ambient pressure (i.e. at 0.1 MegaPascal) of equal to or less than 0° C., more preferably equal to or less than minus 5 degrees Celsius (−5° C.), even more preferably equal to or less than minus 10 degrees Celsius (−10° C.) and still more preferably equal to or less than minus 20 degrees Celsius (−20° C.). Preferably, the water-immiscible displacement fluid has no flash point or a flash point equal to or more than 60° C., even more preferably equal to or more than 80° C. and still more preferably equal to or more than 100° C. Such a flashpoint may for example be determined by ASTM method no. ASTM D93.

Clearly, for the displacement liquid to easily flow through the interstitial space of the reactor, it is preferred that the viscosity is not unduly high. Hence, it is preferred that the water-immiscible displacement liquid has a viscosity at 20° C. of equal to or less than 5 centipoise (cP), more preferably equal to or less than 4.0 cP and most preferably equal to or less than 2 cP. Such viscosity may for example be determined by ASTM method no. ASTM D445-17a. Additionally, it is preferred that the water-immiscible displacement fluid is a liquid having a density equal to or less than 1200 kilograms per cubic meter (kg/m³), even more preferable a liquid having a density equal to or less than 1000 kg/m³ and still more preferably a liquid having a density equal to or less than 800 kg/m³. Such density may for example be determined by ASTM method no. ASTM D1217-15. Preferably, the displacement fluid is essentially water-free, and preferably essentially immiscible with an aqueous hydrochloric acid solution and/or an aqueous first hydrolysate product solution and/or an aqueous second hydrolysate product solution as described herein. Preferably, the water-immiscible displacement liquid comprises or consists of one or more alkanes, more preferably one or more alkanes having in the range from equal to or more than 5 to equal to or less than 20 carbon atoms, even more preferably an alkane having in the range from equal to or more than 6 to equal to or less than 16 carbon atoms. The alkanes may be cyclic or non-cyclic. Most preferably, the water-immiscible displacement liquid comprises or consists of one or more alkanes chosen from the group consisting of cyclic hexane, normal hexane, iso-hexane and other hexanes, normal heptane, iso-heptane and other heptanes, normal octane, iso-octane and other octanes, normal nonane, iso-nonane and other nonanes, normal decane, iso-decane and other decanes, normal undecane, iso-undecane and other undecanes, normal dodecane, iso-dodecane and other dodecanes, normal tridecane, iso-tridecane and other tridecanes, normal tetradecane, iso-tetradecane and other tetradecanes, normal pentadecane, iso-pentadecane and other pentadecanes, normal hexadecane, iso-hexadecane and other hexadecanes.

The processes of the present invention will work well if in a reactor packed with particulate matter there is still some interstitial space, through which the hydrochloric acid and displacement fluid can percolate. For such, in the present invention it is preferred that the reactor comprising said particulate matter and interstitial space has a porosity calculated as V_{interstitial space}/V_{bulk} of between 0.1 and 0.5, preferably said porosity is between 0.2 and 0.4, wherein V_{bulk}=V_{interstitial space}+V_{particulates}, and V is the volume in such.

Typically, in the process according to the present invention the particulate matter comprising hemicellulose, cellulose and lignin is preferably particulate matter of vegetable biomass. The particulate matter may conveniently be washed, dried, roasted, torrefied and/or reduced in particle size before it is used as a feedstock in the process according to the invention. The particulate matter may conveniently be supplied or be present in a variety of forms, including chips, pellets, powder, chunks, briquettes, crushed particles, milled particles, ground particles or a combination of two or more of these. Suitable examples of such particulate matter include wood chips, preferably woodchips from softwood or rubberwood.

### EXAMPLES

### Example 1

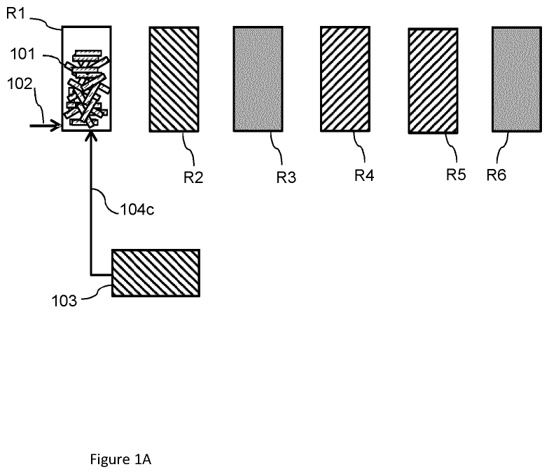

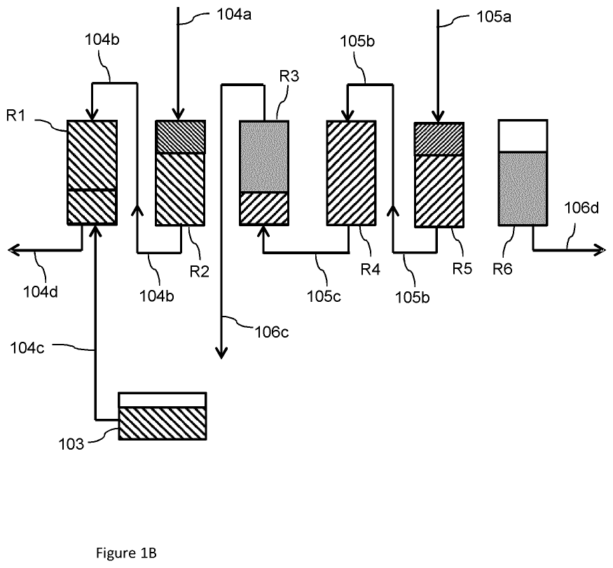

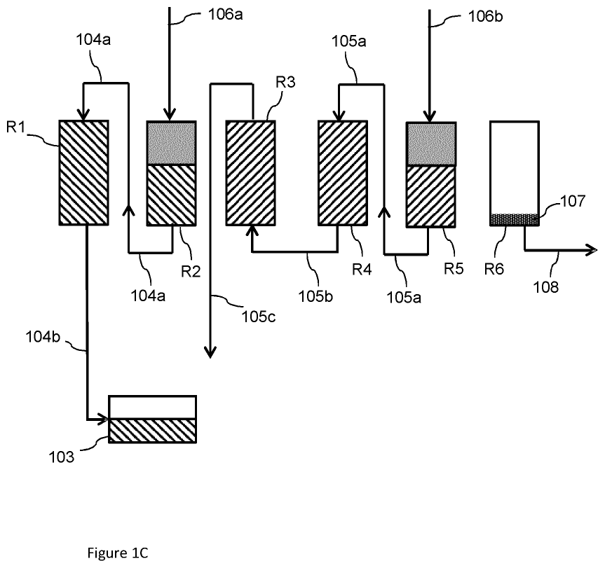

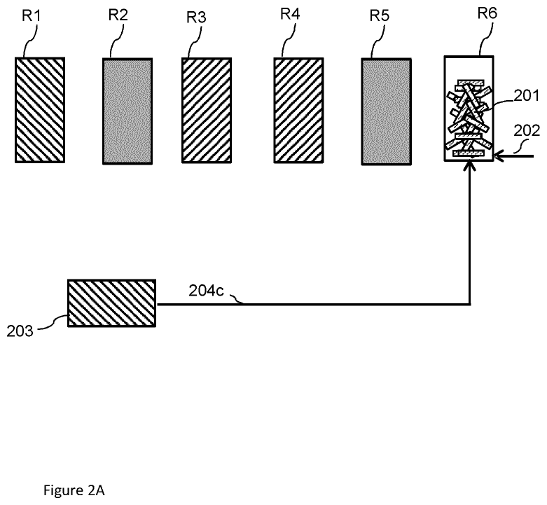

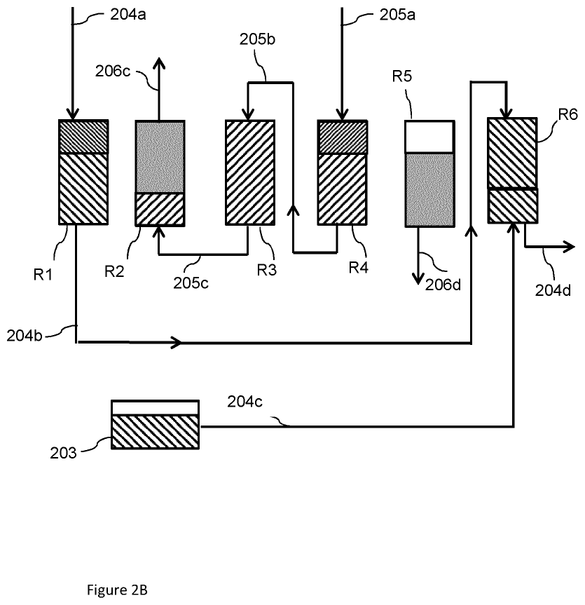

Non-limiting FIGS. 1A, 1B, 1C, 2A and 2B illustrate an example of a process of hydrolysing particulate matter containing hemicellulose, cellulose, and lignin, with hydrochloric acid. A brief description of the figures of this example:

FIGS. 1A, 1B and 1C illustrate a first cycle, starting at a time “t”, of a process according to the invention.

FIGS. 2A and 2B illustrate a second subsequent cycle, starting at a time “t+8 hours”, of the same process as FIGS. 1A, 1B and 1C.

The illustrated process is carried out in a reactor sequence of 6 hydrolysis reactors (R1 to R6). The hydrolysis reactors are operated at a temperature of 20° C. and a pressure of 0.1 Mega Pascal. The process is operated in a sequence of cycles, each cycle being carried out within a 8 hour cycle period.

FIG. 1A illustrates the start of a new cycle. At the start of a new cycle, dried wood chips (101) have just been loaded into reactor (R1) via solid inlet line (102). Reactor (R2) contains an intermediate prehydrolysate solution and a solid material containing cellulose and lignin. The hemicellulose is already at least partly hydrolysed. Reactor (R3) contains a displacement fluid (such as for example iso-octane) and a solid material containing cellulose and lignin. Reactors (R4) and (R5) each contain an intermediate hydrolysate solution. The intermediate hydrolysate solution in reactor (R4) can contain a higher amount of saccharides than the intermediate hydrolysate solution in reactor (R5), as explained below. In addition reactors (R4) and (R5) contain a solid material containing lignin. The cellulose is already at least partly hydrolysed. Reactor (R6) contains a displacement fluid (such as for example iso-octane) and a residue. The residue is a solid material containing lignin.

As illustrated in FIG. 1B, during a first part of the cycle, reactor (R1) is flooded with a plug (104c) of intermediate prehydrolysate solution coming from a storage vessel (103), a plug (104a) of fresh first aqueous hydrochloric acid solution is introduced to reactor (R2), a plug (105a) of fresh second aqueous hydrochloric acid solution is introduced to reactor (R5) and a plug (106d) of displacement fluid is drained from reactor (R6).

After reactor (R1) has been flooded with a plug (104c when going into R1, 104d when being pushed out of R1) of intermediate prehydrolysate solution coming from a storage vessel (103), a plug (104a) of fresh first aqueous hydrochloric acid solution, having a hydrochloric acid concentration of 37.0 wt. % and containing essentially no saccharides yet, is introduced into reactor (R2), thereby pushing forward a plug (104b) of intermediate pre-hydrolysate solution, containing hydrochloric acid in a concentration of about 37.0 wt. %, but also containing already some saccharides (i.e. saccharides derived from solid material that was residing in reactor (R2)), from reactor (R2) into reactor (R1).The plug (104b) of intermediate pre-hydrolysate solution, pushes the plug (104d) out from reactor (R1). Plug (104d) previously contained intermediate pre-hydrolysate solution, but has now taken up sufficient saccharides and has become a final first hydrolysate product solution. Such final first hydrolysate product solution can suitably be forwarded to one or more subsequent processes or devices, where optionally hydrochloric acid could be removed from the pre-hydrolysate solution and recycled.

During the same first part of the cycle, a plug (105a) of fresh second aqueous hydrochloric acid solution, having a hydrochloric acid concentration of 42.0 wt. % and containing essentially no saccharides yet, is introduced into reactor (R5), thereby pushing forward a plug (105b) of intermediate hydrolysate solution, containing hydrochloric acid in a concentration of about 42.0 wt. %, but also containing already some saccharides (i.e. derived from the solid material that was residing in reactor (R5)), from reactor (R5) into reactor (R4). This plug (105b) in its turn pushes forward a second plug (105c) of intermediate hydrolysate solution, containing hydrochloric acid in a concentration of about 42.0 wt. %, but also containing saccharides (i.e. derived from solid material that was residing in previous reactors), from reactor (R4) into reactor (R3). Whilst being pushed from reactor (R5) into reactor (R4) and further into reactor (R3), the intermediate hydrolysate solution absorbs more and more saccharides from the solid material remaining in such reactors from previous stages. The saccharide concentration of the intermediate hydrolysate solution advantageously increases, thus allowing a saccharide concentration to be obtained, that is higher than the saccharide concentration obtained in a batch-process.

The plug (105c) of intermediate hydrolysate solution being pushed from reactor (R4) into reactor (R3), pushes a plug (106c) of displacement fluid out of reactor (R3).

During this same first part of the cycle, further a plug (106d) of displacement fluid is drained from reactor (R6), leaving behind a residue containing lignin.

During a second part of the cycle, as illustrated by FIG. 1C, a plug (106a) of displacement fluid is introduced into reactor (R2). This plug (106a) may or may not contain parts of the plug (106c) of displacement fluid that was pushed out of reactor (R3). Advantageously, the volume of displacement fluid in plug (106a) can be adjusted, for example by adding more or less displacement fluid, to compensate for volume losses due to the reduction of solid material volume. This allows one to ensure that all reactors remain sufficiently filled with volume and it allows one to maintain a sufficient flowrate.

The plug (106a) of displacement fluid being introduced in reactor (R2), suitably pushes forward plug (104a) that was residing in reactor (R2). Plug (104a), previously contained merely fresh first aqueous hydrochloric acid solution, but has in the meantime taken up saccharides from the solid material in reactor (R2) and has become an intermediate pre-hydrolysate solution. Plug (104a) is pushed out of reactor (R2) into reactor (R1), thereby pushing forward plug (104b) of intermediate pre-hydrolysate solution out of reactor (R1) into storage vessel (103) as illustrated in FIG. 1C.

In addition, suitably, a plug of displacement fluid (106b) is introduced into reactor (R5). The plug (106b) of displacement fluid being introduced in reactor (R5), suitably pushes forward plug (105a) that was residing in reactor (R5). Plug (105a), previously contained merely fresh second aqueous hydrochloric acid solution, but has in the meantime taken up saccharides from the solid material in reactor (R5) and has become an intermediate hydrolysate solution. Plug (105a) is pushed out of reactor (R5) into reactor (R4), thereby pushing forward plug (105b) of intermediate pre-hydrolysate solution out of reactor (R4) into reactor (R3). The plug (105b) of intermediate pre-hydrolysate solution, pushes forward plug (105c) that was residing in reactor (R3). Plug (105c), previously contained intermediate hydrolysate solution, but has now taken up sufficient saccharides and has become an aqueous second hydrolysate product solution. Such second hydrolysate product solution can also be referred to as a hydrolysate product solution. Plug (105c) of second hydrolysate product solution is pushed out from reactor (R3). Such second hydrolysate product solution can suitably be forwarded to one or more subsequent processes or devices, where optionally hydrochloric acid could be removed from the hydrolysate solution and recycled.

During this same second part of the cycle, residue (107) containing lignin can suitably be removed from reactor (R6) via solid outlet line (108) and reactor (R6) can be loaded with a new batch of dried wood chips (shown as (201) in FIG. 2A).

The cycle has now been completed and all reactors have shifted one position in the reactor sequence. That is:
reactor (R6) has now shifted into the position previously occupied by reactor (R1);
reactor (R1) has now shifted into the position previously occupied by reactor (R2);
reactor (R2) has now shifted into the position previously occupied by reactor (R3);
reactor (R3) has now shifted into the position previously occupied by reactor (R4);
reactor (R4) has now shifted into the position previously occupied by reactor (R5); and
reactor (R5) has now shifted into the position previously occupied by reactor (R6).

As indicated, the above cycle takes about 8 hours. A subsequent cycle can now be started.

The situation wherein all reactors have shifted one position has been illustrated in FIG. 2A. FIG. 2A illustrates the start of a subsequent cycle, at a time “t+8 hours”. The dried wood chips in what was previously reactor (R6) and is now reactor (R1) can be flooded with a plug (204c) of intermediate pre-hydrolysate solution withdrawn from the storage vessel (103). This is the same intermediate pre-hydrolysate solution that was stored in such storage vessel (103) as plug (104b) of intermediate pre-hydrolysate solution in the second part of the previous cycle, and illustrated in FIG. 1C. The subsequent cycle can be carried out in a similar manner as described above for the preceding cycle. Such is illustrated in FIG. 2B, where numerals (201), (202), (204a-d), (205a-c) and (206a-d) refer to features similar to the features referred to by numerals (101), (102), (104a-d), (105a-c) and (106a-d) in FIG. 1B.

It is noted that all pre-hydrolysate and hydrolysate solutions in the above examples are suitably aqueous hydrolysate solutions, respectively aqueous pre-hydrolysate solutions.

1. A process for preparing alkylene glycol from particulate matter comprising hemicellulose, cellulose and lignin, which process comprises the steps of subjecting a reactor comprising particulate matter comprising hem icellulose, cellulose and lignin and interstitial space to the following steps:
a. feeding to said reactor a first hydrochloric acid solution to hydrolyse at least part of the hemicellulose of said particulate matter by contacting the particulate matter with said first aqueous hydrochloric acid solution, which first aqueous hydrochloric acid solution has a hydrochloric acid concentration of between 30 wt. % and 42 wt. %, based on the weight amount of water and hydrochloric acid in such first aqueous hydrochloric acid solution, yielding a first remaining particulate matter and a first aqueous hydrolysate product solution;
b. feeding to said reactor a water-immiscible displacement fluid thereby displacing at least part of said first aqueous hydrolysate product solution from the interstitial space with said water-immiscible displacement fluid;
c. feeding to said reactor a second hydrochloric acid solution to hydrolyse at least part of the cellulose of the first remaining particulate matter by contacting the first remaining particulate matter with said second hydrochloric acid solution, which second hydrochloric acid solution has a hydrochloric concentration of between 40% and 51%, based on the weight amount of water and hydrochloric acid in the second hydrochloric acid solution whilst said second hydrochloric acid solution has a hydrochloric acid concentration which is the same or higher than the first hydrochloric acid solution added in step a., yielding a second remaining particulate solid material and a second aqueous hydrolysate product solution;
d. subjecting said first aqueous hydrolysate product and/or second aqueous hydrolysate product to a catalytic conversion with hydrogen and in the presence of a catalyst system to a product comprising one or more alkylene glycols,
wherein the catalytic conversion in step d. comprises a catalyst system comprising a tungsten compound, and at least one hydrogenolysis metal selected from the groups 8, 9 or 10 of the Periodic Table of the Elements.

2. The process according to claim 1, wherein in the catalyst system the molar ratio of moles tungsten to moles hydrogenolysis metal is equal to or more than 1:1

3. The process according to claim 1, wherein the tungsten compound has an oxidation state of at least 2+.

4. The process Process according to claim 1, wherein the tungsten compound is selected from the group consisting of: sodium tungstate (Na₂WO₄), tungstic acid (H₂WO₄), ammonium tungstate, ammonium metatungstate, ammonium paratungstate, tungstate compounds comprising at least one Group 1 or 2 element, metatungstate compounds comprising at least one Group 1 or 2 element, paratungstate compounds comprising at least one Group 1 or 2 element, tungsten oxide (WO₃), heteropoly compounds of tungsten, and combinations thereof.

5. The process according to claim 1, wherein the hydrogenolysis metal from groups 8, 9 or 10 of the Periodic Table of the Elements is selected from the group consisting of Cu, Fe, Ni, Co, Pd, Pt, Ru, Rh, Ir, Os and combinations thereof.

6. The process according to claim 1, wherein the hydrogenolysis metal from the groups 8, 9 or 10 of the Periodic Table of the Elements is present in the form of a catalyst supported on a carrier.

7. The process according to claim 6, wherein the carrier is selected from the group supports, consisting of activated carbon, silica, alumina, silica-alumina, zirconia, titania, niobia, iron oxide, tin oxide, zinc oxide, silica-zirconia, zeolitic aluminosilicates, titanosilicates, magnesia, silicon carbide, clays and combinations thereof.

8. The process according to claim 1, wherein the catalyst system comprises ruthenium on activated carbon.

9. The process according to claim 1, wherein step d. is carried out in a reactor system comprising a continuously stirred tank reactor (CSTR).

10. The process according to claim 1, wherein the alkylene glycol is ethylene glycol and/or propylene glycol.

11. The process according to claim 1, wherein the feeding to said reactor of said second hydrochloric acid solution in step c. displaces at least part of the water-immiscible displacement fluid from step b., thereby effecting removal of at least part of said water-immiscible displacement fluid from the interstitial space.

12. The process according to claim 1, wherein after step c. and prior to step d. there is an additional step of transferring the first and/or second aqueous hydrolysate product to a continuously stirred tank reactor for effecting the catalytic conversion step d.

13. The process according to claim 1, wherein the water-immiscible displacement fluid is a liquid that has a solubility in water of less than 3 g liquid per litre of water, at 20° C. and atmospheric pressure, preferably having a solubility of less than 2 g/L, even more preferably less than 1 g/L.

14. The process according to claim 13, wherein the non-aqueous displacement liquid comprises or consists of one or more alkanes chosen from the group consisting of cyclic hexane, normal hexane, iso-hexane and other hexanes, normal heptane, iso-heptane and other heptanes, normal octane, iso-octane and other octanes, normal nonane, iso-nonane and other nonanes, normal decane, iso-decane and other decanes, normal undecane, iso-undecane and other undecanes, normal dodecane, iso-dodecane and other dodecanes, normal tridecane, iso-tridecane and other tridecanes, normal tetradecane, iso-tetradecane and other tetradecanes, normal pentadecane, iso-pentadecane and other pentadecanes, normal hexadecane, iso-hexadecane and other hexadecanes.
